(19) United States
(12) Patent Application Publication
(10) Pub. No.: US 2011/0282277 A1
(43) Pub. Date: Nov. 17, 2011

(54) DEVICE FOR CONTROLLING INJECTION OF RINGER'S SOLUTION

(76) Inventor: Hwa Joong Kim, Gwangju (KR)

(21) Appl. No.: 13/132,869

(22) PCT Filed: Dec. 9, 2009
(86) PCT No.: PCT/KR2009/007362

§ 371 Date: Jul. 21, 2011

(30) Foreign Application Priority Data
Dec. 9, 2008 (KR) 10-2008-0124529
Sep. 9, 2009 (KR) 10-2009-0085040

(51) Int. Cl. A61M 5/168

(52) U.S. Cl. 604/65

(57) ABSTRACT

Disclosed is a Ringer solution infusion controlling apparatus which enables the infused amount and infusion state of a Ringer solution to be easily monitored by an administrator and which automatically blocks infusion of the Ringer solution when the infusion of the Ringer solution is completed. The Ringer solution infusion controlling apparatus includes a body coupled to a chamber receiving a Ringer's solution from a Ringer bottle in which the Ringer's solution is stored and ejecting the Ringer's solution in a state in which the Ringer's solution is maintained at a constant level by first coupling means, a sensing ball provided in the chamber and floating on the Ringer's solution, and elevating according to the level of the Ringer's solution in the chamber, an elevation sensor installed in the body and sensing elevation of the sensing ball, a Ringer's solution transfer blocking unit installed in the body and having a Ringer hose passing therethrough, the Ringer hose allowing the Ringer's solution ejected from the chamber to be transferred, and blocking transfer of the Ringer's solution by compressing the Ringer hose when it is detected by the elevation sensor that the sensing ball deviates from a predetermined range, and a wireless transmitter installed in the body and wirelessly transmitting a Ringer's solution transfer blocking signal to a nurse's room when the transfer of the Ringer's solution is blocked by the Ringer's solution transfer blocking unit.

### TECHNICAL FIELD

The present invention relates to a Ringer solution infusion controlling apparatus, and more particularly, to a Ringer solution infusion controlling apparatus which enables the infused amount and infusion state of a Ringer solution to be easily monitored by an administrator and automatically blocks infusion of the Ringer solution when the infusion of the Ringer solution is completed.

### BACKGROUND ART

In general, a Ringer's solution is a dextrose-containing fluid used to directly inject dextrose to the blood vessel of a patient who has poor health or is on a fast due to surgery. In order to continuously administer the Ringer's solution, a Ringer's solution infusion apparatus has been used.

A conventional Ringer's solution injecting apparatus includes a Ringer bottle in which a Ringer's solution is stored, an ejection needle coupled to the Ringer bottle, a pressure supply unit allowing the Ringer's solution ejected through the ejection needle using a pressure based on a phase difference, and an injection needle inserted into a patient's blood vessel.

The pressure supply unit includes a chamber receiving the Ringer's solution discharged from the ejection needle and maintaining a predetermined level of the Ringer's solution, a floating ball positioned in the chamber, floating on the solution stored in the chamber and lowering to close an outlet of the chamber when the solution is exhausted, and preventing the blood from flowing backward from the patient's blood vessel, and a Ringer's solution supply controller controlling an amount of the Ringer's solution discharged by controlling a cross-sectional area of the Ringer hose guiding the Ringer's solution discharged from the chamber to the injection needle.

In the conventional Ringer's solution injecting apparatus, when the Ringer's solution of the Ringer bottle is infused into the patient and used up, the floating ball provided in the chamber closes the outlet of the chamber, thereby preventing the blood from flowing backward from the patient's blood vessel by automatically blocking infusion of the Ringer's solution.

However, if the outlet of the chamber is not completely closed by the floating ball, a dangerous emergent situation, including backward flow of blood, infusion of the air into the patient's blood vessel, may occur.

Accordingly, the patient or nurse is forced to check frequently a state or amount of the Ringer's solution infused for a long time, which is quite inconvenient and burdensome.

### DISCLOSURE OF THE INVENTION

In order to overcome the above-mentioned shortcomings, the present invention provides a Ringer's solution infusion controlling apparatus which can detect a state of a Ringer's solution infused in an unmanned manner, automatically block infusion of the Ringer's solution when infusion is completed, and notify a patient or a nurse of the completion of infusion.

The present invention also provides a Ringer's solution infusion controlling apparatus configured to monitor an infusion state of a Ringer's solution and set an infusion amount of the Ringer's solution.

According to an aspect of the invention, there is provided a Ringer's solution infusion controlling apparatus including a body coupled to a chamber receiving a Ringer's solution from a Ringer bottle in which the Ringer's solution is stored and ejecting the Ringer's solution in a state in which the Ringer's solution is maintained at a constant level by first coupling means, a sensing ball provided in the chamber and floating on the Ringer's solution, and elevating according to the level of the Ringer's solution in the chamber, an elevation sensor installed in the body and sensing elevation of the sensing ball, a Ringer's solution transfer blocking unit installed in the body and having a Ringer hose passing therethrough, the Ringer hose allowing the Ringer's solution ejected from the chamber to be transferred, and blocking transfer of the Ringer's solution by compressing the Ringer hose when it is detected by the elevation sensor that the sensing ball deviates from a predetermined range, and a wireless transmitter installed in the body and wirelessly transmitting a Ringer's solution transfer blocking signal to a nurse's room when the transfer of the Ringer's solution is blocked by the Ringer's solution transfer blocking unit.

The Ringer's solution transfer blocking unit may include electromagnets positioned in the body to be opposed to each other via the body with the Ringer hose disposed therebetween, installed to be slidable toward the Ringer hose via the body, receiving external power to generate a magnetic force, sliding toward the Ringer hose by an attraction and pressing the Ringer hose, a control unit receiving a detection signal from the elevation sensor to detect presence or absence of the sensing ball within the predetermined range and selectively supply the external power to the electromagnets, and an elastic member installed in the body and allowing the electromagnets to be restored into their original positions by an elastic force, when supply of the external power to the electromagnets is blocked.

The Ringer solution infusion controlling apparatus may further include second coupling means provided above the body and coupling and fixing the body to a supply hose supplying the chamber with the Ringer's solution from the Ringer bottle, a speaker installed in the body, controlled by the control unit having received a detection signal from the elevation sensor, the detection signal indicating whether the sensing ball is within a predetermined range or not, and selectively generating an warning sound, a first storage battery installed in the body and supplying power to the wireless transmitter, the electromagnets, and the speaker, a second storage battery positioned outside the body, connected to a power supply jack installed in the body and supplying power to the wireless transmitter, the electromagnets, the speaker, a residual power measuring unit installed in the body and measuring residual power of the first storage battery, an indicator lamp controlled by the control unit, and when a residual power level of the first storage battery, measured by the residual power measuring unit, is a predetermined value, receiving power from the second storage battery and emitting light to indicate power consumption of the first storage battery, and a blocking switch installed in the body and forcibly blocking power supply from the first and second storage batteries to the electromagnets, wherein when a residual power level of the first storage battery, measured by the residual power measuring unit, is a predetermined value, the control unit receives a detection signal from the elevation sensor, the detection signal indicating whether the sensing ball is within a predetermined range or not, and controls the power from the second storage battery to be selectively supplied to the wireless transmitter, the electromagnets, the speaker.

According to another aspect of the present invention, there is provided a Ringer solution infusion controlling apparatus including a first unit supporting a Ringer's solution storage container and including a sensor sensing an amount of the Ringer's solution stored in the Ringer's solution storage container, a second unit connected to the Ringer's solution storage container to support a predetermined region of a transfer tube for transferring the Ringer's solution and including an infusion blocking unit blocking movement of the Ringer's solution passing through the transfer tube, a control unit receiving sensing information of the sensor, driving the infusion blocking unit and including a transmitter transmitting the sensing information of the sensor and a signal to indicate whether the infusion blocking unit is driven or not, and a display unit installed in an administration room or a nurse room and including a receiver receiving the transmission signal of the transmitter and a display indicating a signal received from the receiver.

The first unit may include a hanger unit having a fixing protrusion formed at one side of an upper portion of the hanger unit to hang the Ringer's solution storage container, the sensor is installed in the hanger unit to be movable up and down to detect a level of the Ringer's solution contained in the Ringer's solution storage container hung on the hanger unit to set an infusion amount of the Ringer's solution, the second unit includes a support plate, and a tube support unit installed in the support plate and supporting a predetermined region of the transfer tube so as to extend in a linearly movable state, the infusion blocking unit is a solenoid valve blocking movement of the Ringer's solution by shutting down the conduit line of the transfer tube supported on the tube support unit.

The support plate may further include a camera that photographs a patient and patient's surroundings to monitor the patient's condition by images through the display unit, and a call button provided to allow the patient to call a nurse, and the photographed image from the camera and a call signal from the call button are transmitted to the display unit through the control unit.

Here, the hanger unit may have a sliding hole extending up and down and penetrating inner and outer surfaces thereof contacting the Ringer's solution storage container hung on the fixing protrusion. In addition, the sensor may include an inner cylinder member including a first cylinder unit passing through the sliding hole and extending and having a hollow lengthwise and a first fixing flange provided at one end of the first cylinder unit and having an outer diameter greater than a width of the sliding hole to have a surface contacting the inner surface of the hanger unit, a screw coupling member threaded with the hollow of the inner cylinder member, a sensor member extending from the screw coupling member toward the inside of the hanger unit and detecting a level of the Ringer's solution stored in the Ringer's solution storage container, an extension rod extending from the screw coupling member toward the outside of the hanger unit, an outer cylinder member including a second cylinder unit slidably surrounding the first cylinder unit from the outside of the hanger unit, a second fixing flange provided at one end of the second cylinder unit and having a surface contacting the outer surface of the hanger unit, and a cover unit coupled to the other end of the second cylinder unit and having a passing-through hole through which the extension rod can pass, a support flange formed at an end of the extension rod extending through the cover unit and protruding, and having an outer diameter larger than that of the extension rod, and an elastic member having one and the other ends supported by the support flange and the cover unit, respectively and installed to surround the extension rod to be elastically biased so as to pressurize outer and inner surfaces of the hanger unit, thereby fixing the inner cylinder member and the outer cylinder member on the hanger unit hat the first and second fixing flanges.

### ADVANTAGEOUS EFFECTS

As described above, according to the present invention, completion of infusion of a Ringer's solution can be monitored in an unmanned manner to automatically notify a nurse, a patient or a patent's guardian of a replacement time of the Ringer's solution, thereby preventing a medical accident. It is also possible to prevent the Ringer's solution infusion controlling apparatus from malfunctioning in an emergent situation by using an auxiliary storage battery.

In addition, the Ringer's solution infusion controlling apparatus according to the present invention can easily set and adjust an amount of a Ringer's solution to be infused to a patient, thereby preventing the Ringer's solution from being excessively infused. Further, in the Ringer's solution infusion controlling apparatus according to the present invention, patient administration can be easily made, thereby reducing nurses' work burden.

### BRIEF DESCRIPTION OF THE DRAWINGS

The objects, features and advantages of the present invention will be more apparent from the following detailed description in conjunction with the accompanying drawings, in which:

FIG. 1 illustrates a state in which a Ringer's solution infusion controlling apparatus according to a first embodiment of the present invention is used;

FIG. 2 illustrates a structure of the Ringer's solution infusion controlling apparatus shown in FIG. 1;

FIG. 3 illustrates a state in which the Ringer's solution infusion controlling apparatus shown in FIG. 1 is assembled to a chamber,

FIG. 4 is a control block diagram of the Ringer's solution infusion controlling apparatus shown in FIG. 1;

FIG. 5 is a perspective view of a Ringer's solution according infusion controlling apparatus according to a second embodiment of the present invention;

FIG. 6 is an exploded perspective view of a first unit shown in FIG. 5;

FIG. 7 is a cross-sectional view of the first unit shown in FIG. 5;

FIG. 8 is a perspective view of a second unit in FIG. 5;

FIGS. 9 and 10 are cross-sectional views illustrating a state in which an infusion blocking unit is driven;

FIG. 11 is a block diagram illustrating a driving mechanism of the Ringer's solution infusion controlling apparatus shown in FIG. 5;

FIG. 12 is an exploded cut-away perspective view illustrating an embodiment in which a sensor is installed in a chamber;

FIG. 13 is a perspective view illustrating another embodiment of a second unit including an infusion speed adjusting device; and

FIG. 14 is a cross-sectional view of illustrating still another embodiment of an infusion speed adjusting device.

### BEST MODE FOR CARRYING OUT THE INVENTION

Hereinafter, embodiments of the present invention will be described in detail with reference to the accompanying drawings.

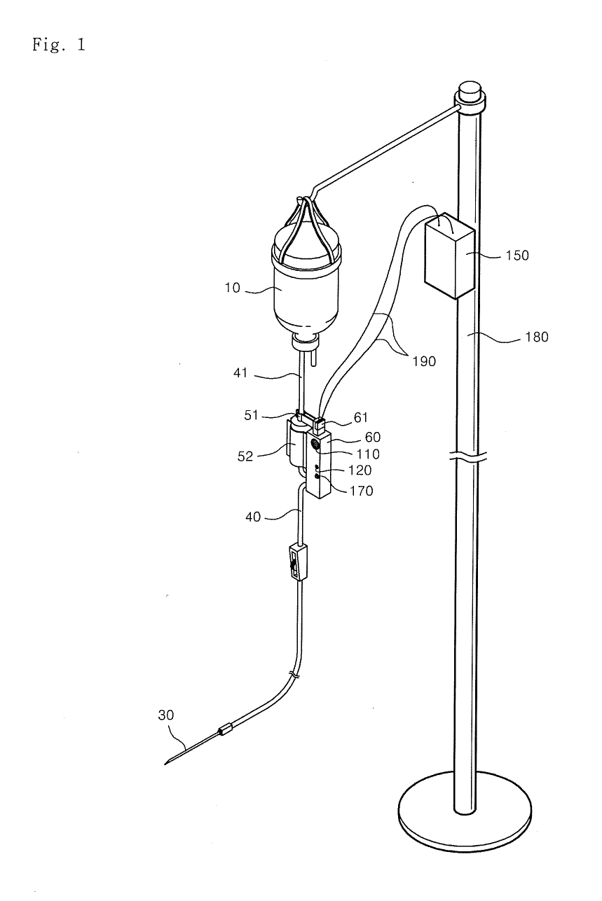

A Ringer's solution infusion controlling apparatus according to a first embodiment of the present invention will be described with reference to FIGS. 1 to 3.

Referring to FIG. 1, the first embodiment of the present invention is directed to a Ringer's solution infusion controlling apparatus including a Ringer bottle 10 in which a Ringer's solution is stored, a chamber 20 receiving the Ringer's solution from the Ringer bottle 10 and ejecting the Ringer's solution in a state in which the Ringer's solution is maintained at a constant level, and a Ringer hose 40 connecting the chamber 20 and an injection needle 30 inserted into patient's blood vessel and allowing the Ringer's solution to pass therethrough, thereby monitoring completion of infusion of the Ringer's solution by detecting a level of the Ringer's solution stored in the chamber 20.

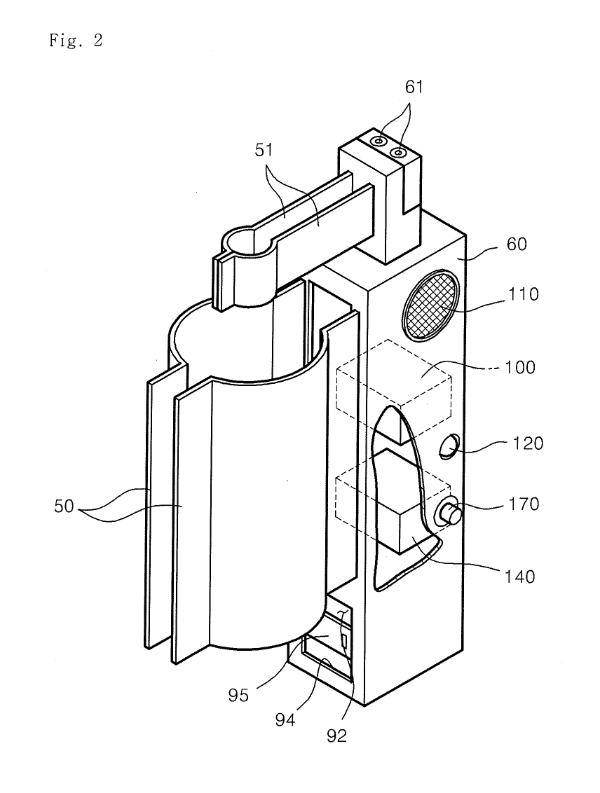

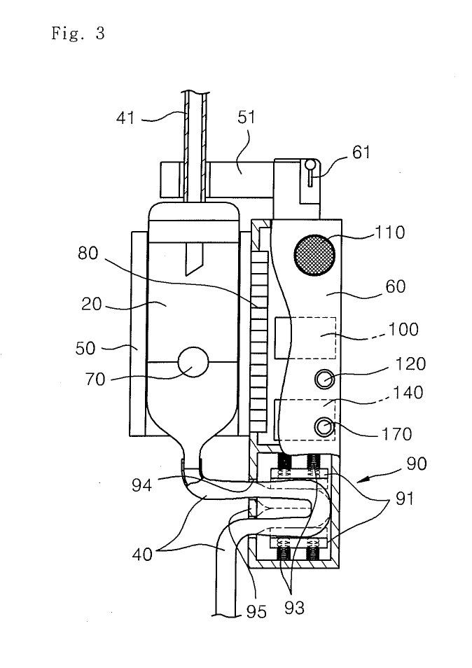

Referring to FIGS. 2 and 3, the Ringer's solution infusion controlling apparatus further includes a body 60, a sensing ball 70, an elevation sensor 80, and a Ringer's solution transfer blocking unit 90. The body 60 is coupled to the chamber 20 by first coupling means 50. The sensing ball 70 is provided within the chamber 20 and floats on the Ringer's solution to elevate according to the level of the Ringer's solution. The elevation sensor 80 is installed in the body 60 and detects elevation of the sensing ball 70. The Ringer's solution transfer blocking unit 90 is installed in the body 60, allows the Ringer hose 40 to pass by the body 60, and blocks transfer of the Ringer's solution by compressing the Ringer hose 40 if the elevation sensor 80 detects that the sensing ball 70 deviates from a predetermined range.

The Ringer's solution transfer blocking unit 90 folds the Ringer hose 40 passing by the body 60 by pressurizing the Ringer hose 40 using electromagnets 91 closely contacting each other by attraction, thereby blocking transfer of the Ringer's solution passing by the body 60.

That is to say, a passing space 92 is formed to allow the Ringer hose 40 to pass by the body 60, and the electromagnets 91 facing and spaced apart from each other are installed in the passing space 92. The electromagnets 91 are slidably installed. After the electromagnets 91 closely contact each other by attraction, an elastic member 93 for restoring the electromagnets 91 when no attraction is applied is connected to the electromagnets 91 to then be installed in the passing space 92.

In addition, an inlet 94 into which the Ringer hose 40 is inserted is formed in the body 60. The Ringer hose 40 is inserted into the passing space 92 through the inlet 94, bent in a ‘U’ shape and extracted through the inlet 94, so that it passes via the passing space 92.

In order to prevent the Ringer hose 40 inserted into the passing space 92 from being dislodged from the passing space 92, the inlet 94 includes a door 95 shielding the center of the inlet 94. That is to say, since the Ringer hose 40 inserted in a state in which it is bent in a ‘U’ shape is locked in the door 95, it is possible to prevent the Ringer hose 40 from being dislodged from the passing space 92.

The operation of the electromagnets 91 is carried out by generating a magnetic force by supplying power to the electromagnets 91. The supplying of power to the electromagnets 91 is controlled by a control unit 96, which receives a detection signal from the elevation sensor 80 detecting whether the sensing ball 70 is elevated or not.

If the sensing ball 70 is not detected within the predetermined range, the control unit 96 having received the detection signal allows the power to be supplied to the electromagnets 91.

The Ringer's solution infusion controlling apparatus according to the embodiment of the present invention further includes a wireless transmitter 100 controlled by the control unit 96, a speaker 110, an indicator lamp 120, and an on/off switch 130.

When transfer of the Ringer's solution is blocked by the Ringer's solution transfer blocking unit 90, the wireless transmitter 100 transmits a Ringer's solution transfer blocking signal to the nurse room in a wireless manner to notify the nurse of an infusion status of the Ringer's solution. The wireless transmitter 100 transmits the Ringer's solution transfer blocking signal by patient's room and by patient, thereby allowing the nurse to monitor immediately completion of infusion of the Ringer's solution by patient's room and by patient.

The speaker 110 notifies the patient and patient's guardian of transfer of the Ringer's solution being blocked and infusion of the Ringer's solution being completed.

The indicator lamp 120 is used to indicate that there is no power to be supplied to the electromagnets 91. That is to say, the indicator lamp 120 is used to indicate that the Ringer's solution infusion controlling apparatus according to the embodiment of the present invention is inoperative. More specifically, storage batteries are used as external power source in the Ringer's solution infusion controlling apparatus according to the embodiment of the present invention. The body 60 includes a first storage battery 140 for supplying power to the wireless transmitter 100, the electromagnets 91, and the speaker 110.

In addition, in the embodiment of the present invention, a second storage battery 150 as an auxiliary storage battery is provided in preparation of discharge of the first storage battery 140. The second storage battery 150 is connected to a power supply jack 61 provided in the body 60 to be positioned outside the body 60.

In addition, the body 60 includes a residual power measuring unit 160 for measuring residual power of the first storage battery 140. That is to say, if the residual power level of the first storage battery 140, measured by the residual power measuring unit 160 is a predetermined value, the indicator lamp 120 emits light under the control of the control unit 96.

If the residual power level of the first storage battery 140 is too low a value to pressurize the Ringer hose 40 due to mutual attraction of the electromagnets 91, the control unit 96 controls the on/off switch 130 to be operable so as to connect the wireless transmitter 100, the electromagnets 91 and the speaker 110 to the second storage battery 150 to allow the power of the second storage battery 150 to be supplied to the wireless transmitter 100, the electromagnets 91, and the speaker 110.

Meanwhile, the body 60 includes a blocking switch 170 that forcibly blocking power from being supplied to the electromagnets 91. Once the power is supplied to the electromagnets 91, the supplying of the power to the electromagnets 91 is continuously performed until the blocking switch 170 operates.

In addition to first coupling means 50 for coupling the body 60 to the chamber 20, second coupling means 51 for coupling and fixing the body 60 to a supply hose 41 supplying the Ringer's solution from the Ringer's bottle 10 to the chamber 20 is further provided in an upper portion of the body 60. Clamps may be used as the first and second coupling means 50 and 51.

An infrared sensor, or an ultrasonic sensor may be used as the elevation sensor 80. The elevation sensor 80 may include a plurality of elevation sensors arranged in a predetermined range to detect whether the sensing ball 70 exists in the predetermined range. The elevation sensor 80 may not only to detect the sensing ball 70 but also to detect the Ringer's solution itself when the Ringer's solution is an opaque liquid (e.g., blood for transfusion or a dark colored fluid).

The sensing ball 70 is red so as to be easily detected by the elevation sensor 80, and is made of silicon in an oval shape.

The operation and effects of the aforementioned Ringer's solution infusion controlling apparatus according to the embodiment of the present invention will now be described.

In the embodiment of the present invention, the body 60 is fixed to the chamber 20 by the first and second coupling means 50 and 51, and the second storage battery 150 as an auxiliary storage battery is installed in a hanger pole 180 that suspends the Ringer's bottle 10. The second storage battery 150 is connected to the power supply jack 61 of the body 60 by an electric wire 190.

The Ringer hose 40 connected to the chamber 20 is inserted into the passing space 92 of the body 60 in a ‘U’ shape and fixed by the door 95.

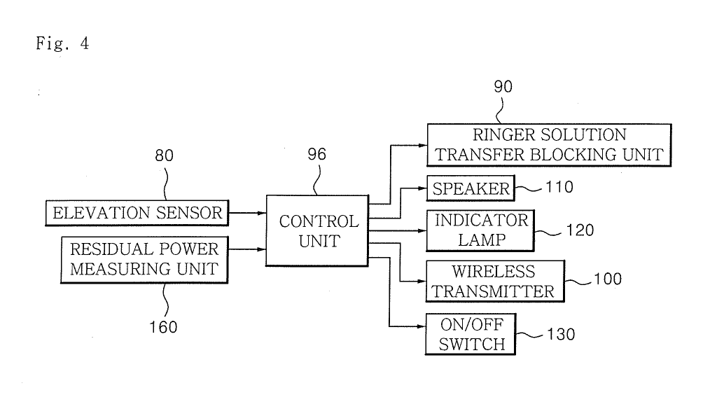

Referring to FIG. 4, if no more Ringer's solution is supplied to the chamber 20 due to consumption of the Ringer's solution in the Ringer's bottle 10, a surface level of the Ringer's solution in the chamber 20 is lowered, making the sensing ball 70 deviate from the predetermined range. This is detected by the elevation sensor 80. The control unit 96 receives a detection signal from the elevation sensor 80 to allow power to be supplied to electromagnets 91 of a Ringer's solution transfer blocking unit 90, and controls a wireless transmitter 100 and a speaker 110 to notify a nurse, a patient and a patent's guardian of a replacement time of Ringer's solution.

If power is supplied to the electromagnets 91, the Ringer hose 40 is pressurized due to mutual attraction of the electromagnets 91 to then be folded. Accordingly, transfer of the Ringer's solution through the Ringer hose 40 is blocked.

Meanwhile, if the residual power of the first storage battery 140 to be supplied to the wireless transmitter 100, the electromagnets 91, and the speaker 110 is not sufficient, the indicator lamp 120 controlled by the control unit 96 notifies the patient or the patient's guardian of a replacement time of the first storage battery 140. In addition, the on/off switch 130 controlled by the control unit 96 allows the power of the second storage battery 150 to be supplied to the electromagnets 91, the wireless transmitter 100, and the speaker 110.

Therefore, according to the embodiment of the present invention, a medical accident, such as backward flow of patient's blood or air induction into patient's blood vessel by malfunction of the Ringer's solution infusion controlling apparatus due to interrupted power supply, can be prevented.

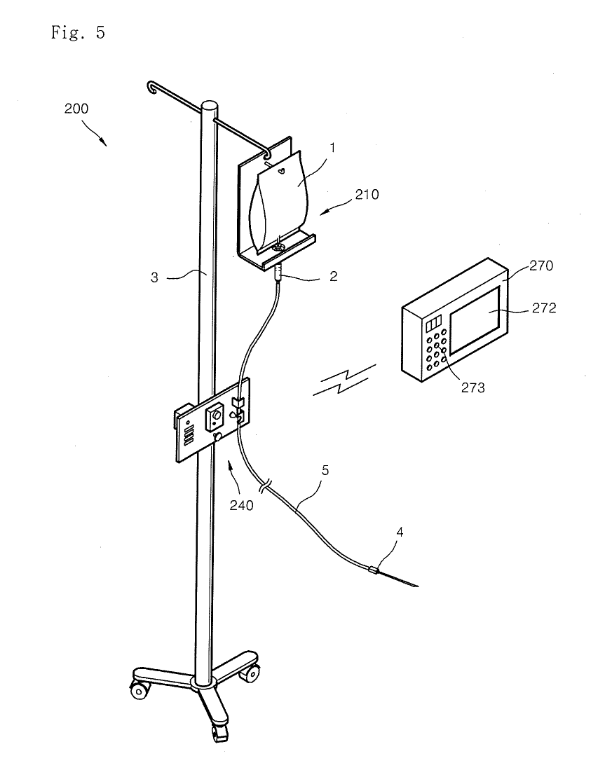

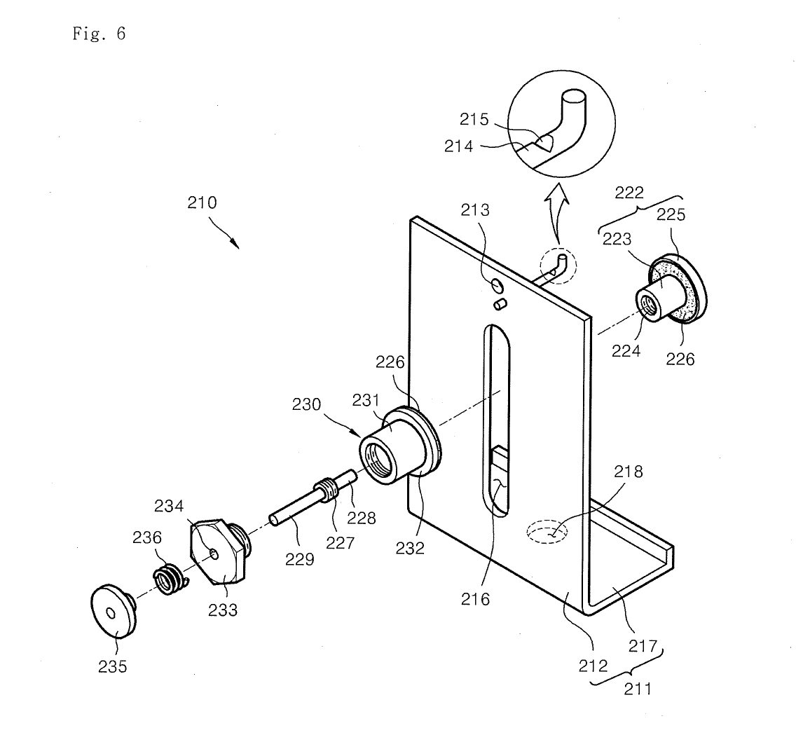

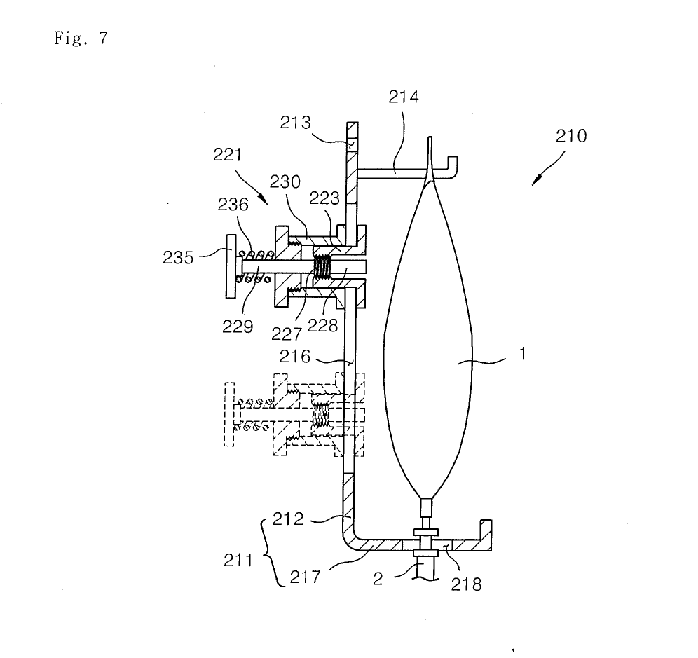

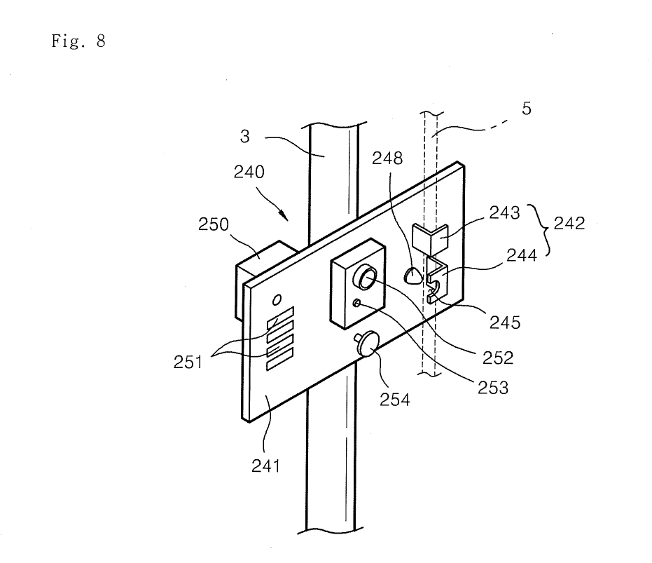

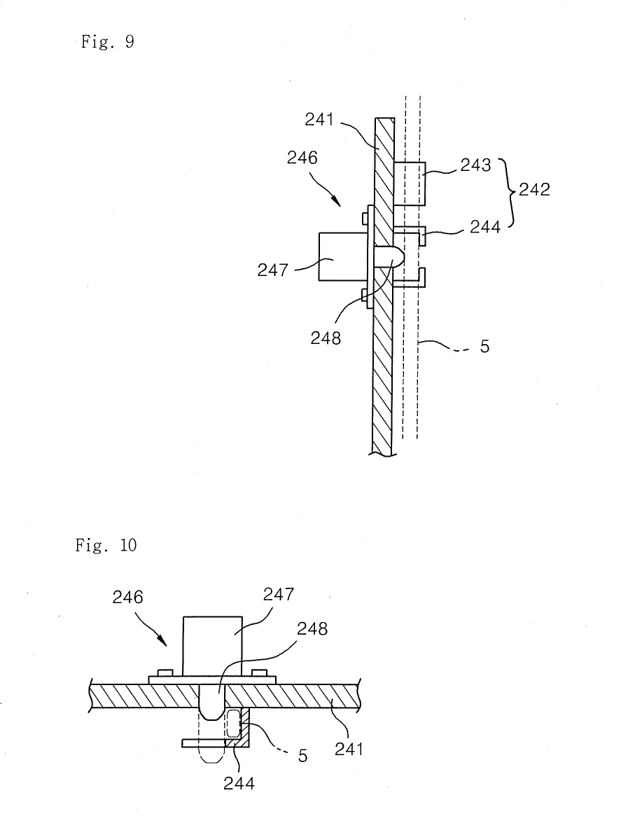

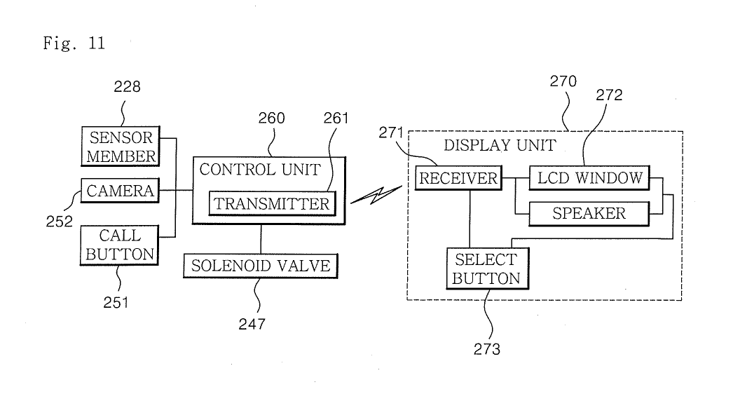

FIGS. 5 to 11 illustrate a Ringer's solution according infusion controlling apparatus 200 according to a second embodiment of the present invention.

Referring to FIGS. 5 to 11, the Ringer's solution infusion controlling apparatus 200 includes a first unit 210 supporting a Ringer's solution storage container 1 and formed to detect an amount of the Ringer's solution stored in the Ringer's solution storage container 1, a second unit 240 supporting a predetermined region of a transfer tube 5 for transferring the Ringer's solution and formed to block a conduit line of the transfer tube 5, a control unit 260 controlling driving of the first and second units 210 and 240 and including a transmitter 261, and a display unit 270 installed in a nurse room, receiving a transmission signal of the control unit 260 and indicating the signal on a display.

The first unit 210 includes a hanger unit 211 supporting the Ringer's solution storage container 1, and a sensor 221 installed in the hanger unit 211 and sensing a level of the Ringer's solution stored in the Ringer's solution storage container 1.

The hanger unit 211 has a vertical panel 212 extending up and down, and a horizontal panel 217 extending at a lower end of the vertical panel 212 in a direction perpendicular to the vertical panel 212. A support hole 213 is formed at an upper portion of the vertical panel 212, and the Ringer's solution storage container 1 is generally hung on a hanger pole 3 using the support hole 213.

For convenience of explanation, a surface of the vertical panel 212, which is adjacent to the Ringer's solution storage container 1, is referred to as an inner surface, and the opposite surface is referred to as an outer surface.

A fixing protrusion 214 extending horizontally by a predetermined length to allow the Ringer's solution storage container 1 to be hung, is formed at an upper portion of the inner surface of the vertical panel 212, and a sliding hole 216 extending through the inner surface and the outer surface is elongated up and down.

The horizontal panel 217 extends horizontally at a lower end of the vertical panel 212. The horizontal panel 217 prevents the Ringer's solution storage container 1 from being dislodged from the hanger unit 211 by being hooked by the horizontal panel 217 without falling down on the ground even when a hooked portion of the Ringer's solution storage container 1 hung on the fixing protrusion 214 is broken.

As described above, stable infusion of the Ringer's solution is achieved even in a contingency by installing the horizontal panel 217. In addition, since the Ringer's solution storage container 1 is positioned lower than a patient, backward flow of blood can be avoided.

A through-hole 218 extending through top and bottom surfaces of the horizontal panel 217 is formed in the horizontal panel 217 to allow the chamber 2 to be infused into the lower end of the Ringer's solution storage container 1. An end of the horizontal panel 217 is bent so as to upwardly extend by a predetermined length in order to support the Ringer's solution storage container 1 in a secured manner when the Ringer's solution storage container 1 is lowered.

A position fixing groove 215 is formed in the fixing protrusion 214. The position fixing groove 215 is inwardly recessed by a predetermined depth, which is for the purpose of fixing the Ringer's solution storage container 1 at a position at which the Ringer's solution storage container 1 is hung on the fixing protrusion 214.

Manufacturing the Ringer's solution storage container 1 in the form of a vinyl pack is quite common these days. When an external shock is applied to the hanger pole 3, for example, when the hanger pole 3 is inadvertently bumped by a passerby, the Ringer's solution storage container 1 on the fixing protrusion 214 may move away from the vertical panel 212. In such a case, it is not easy for the sensor 221 to detect the level of the Ringer's solution. However, when the Ringer's solution storage container 1 closely contacts the inner surface of the vertical panel 212, the vinyl pack of the Ringer's solution storage container 1 may be deformable, which may increase the level of the Ringer's solution.

Therefore, in order to allow the Ringer's solution storage container 1 to be always positioned at an appropriate distance from the vertical panel 212, the position fixing groove 215 is preferably formed for fixing the position of the Ringer's solution storage container 1.

The sensor 221 is installed in the vertical panel 212 so as to be movable up and down. The sensor 221 detects the level of the Ringer's solution storage container 1.

The sensor 221 includes an inner cylinder member 222, an outer cylinder member 230, a screw coupling member 227, a sensor member 228, an extension rod 229, a support flange 235, and an elastic member 236.

The inner cylinder member 222 includes a first cylinder unit 223 shaped of a cylinder having an outer diameter corresponding to a width of the sliding hole 216, and a first fixing flange 225 led to an end of the first cylinder unit 223.

The first cylinder unit 223 has a hollow 224 formed therein, and a screw is formed on its inner surface of the hollow 224 to be threaded with a screw coupling member 227 to be described later. Opposite ends of the first cylinder unit 223 are opened.

The first fixing flange 225 has a centrally formed hole to be connected to the hollow 224. An outer diameter of the first fixing flange 225 is greater than a width of the sliding hole 216. One surface of the first fixing flange 225 exposed toward the first cylinder unit 223 contacts the inner surface of the vertical panel 212. An elastic friction pad 226 is provided in order to increase a frictional force applied to a contact surface between the first fixing flange 225 and the vertical panel 212 and increase an area of the contact surface.

Therefore, the inner cylinder member 222 extends through the sliding hole 216 from the inner surface of the vertical panel 212 to protrude toward the outside of the vertical panel 212 and the first fixing flange 225 is hung on the inner surface of the vertical panel 212.

As described above, the screw coupling member 227 is inserted into the hollow 224 of the inner cylinder member 222 to be threaded with the inner circumferential surface of the inner cylinder member 222. A sensor member 228 is coupled to one side of the screw coupling member 227 and the sensor member 228 is installed to be adjacent with an end of the inner cylinder member 222 to detect the level of the Ringer's solution storage container 1. The sensor member 228 is preferably a capacitive sensor.

The extension rod 229 is connected to the other end of the screw coupling member 227. The extension rod 229 extends toward the outside of the vertical panel 212.

The outer cylinder member 230 includes a second fixing flange 232 contacting the outer surface of the vertical panel 212, a second cylinder unit 231 extending to surround the first cylinder unit 223 from the second fixing flange 232, and a cover unit 233 engaged with an end of the second cylinder unit 231.

Like the first fixing flange 225, the second fixing flange 232 has an outer diameter larger than a width of the sliding hole 216, and an elastic friction pad 226 is formed on its surface contacting the outer surface of the vertical panel 212.

The second cylinder unit 231 surrounds the outer surface of the first cylinder unit 223 and is installed to be slidable by a predetermined distance along the lengthwise direction of the first cylinder unit 223.

The cover unit 233 is formed at the end of the second cylinder unit 231, and a passing-through hole 234 is formed so as to allow the extension rod 229 to pass therethrough. An outer diameter of the cover unit 233 is larger than that of the second cylinder unit 231, so that the edge of the cover unit 233 protrudes from the outer circumferential surface of the second cylinder unit 231.

The support flange 235 is engaged with an end of the extension rod 229 protruding from the cover unit 233 and has a diameter larger than that of the extension rod 229.

An elastic member 236 is formed between the support flange 235 and the cover unit 233 to surround the extension rod 229 and pressurizes the outer cylinder member 230 against the support flange 235 toward the outer surface of the vertical panel 212.

The elastic member 236 makes the outer cylinder member 230 and the inner cylinder member 222 closely contact the outer surface and the inner surface of the vertical panel 212, respectively, thereby fixing the position of the sensor 221. In addition, the elastic member 236 seizes the support flange 235, the cover unit 233 or the second cylinder unit 231 to allow the outer cylinder member 230 to be spaced apart from the vertical panel 212. Then, the sensor 221 is moved to a desired position on the sliding hole 216 to then place the outer cylinder member 230 at a predetermined position spaced apart from the vertical panel. Thus, the outer cylinder member 230 and the inner cylinder member 222 are fixed at positions moved by the elastic member 236.

Therefore, after the sensor member 228 is moved along the sliding hole 216 and then fixed at position, it detects the level of the Ringer's solution when the Ringer's solution reaches at a height of the sensor member 228. Then, the sensor member 228 transmits a detection signal to the control unit 260 to be described later.

The second unit 240 supports a predetermined region of the transfer tube 5 and blocks movement of the Ringer's solution. The second unit 240 includes a support plate 241 coupled to the hanger pole 3, a tube support unit 242 installed at the support plate 241 and supporting the predetermined region of the transfer tube 5 in a linearly movable state, and an infusion blocking unit 246 for stopping infusion of the Ringer's solution by breaking a conduit line of the transfer tube 5.

The tube support unit 242 includes first and second bending members 243 and 244 formed in an inverted-L letter shape and spaced apart from each other in a vertical direction. The first and second bending members 243 and 244 have top and bottom portions opened and open lateral surfaces of the first and second bending members 243 and 244 are opposed to each other in a vertical direction so that the transfer tube 5 inserted into the first and second bending members 243 and 244 is supported so as to extend in a linearly movable state.

In particular, the second bending member 244 has an inlet groove 245 formed to allow the movement member 248 to advance and retreat to/from a plate extending in parallel with a supporting surface of the support plate 241.

The infusion blocking unit 246 includes a solenoid valve 247 installed on a rear surface of the support plate 241. The solenoid valve 247 is driven by a driving signal of the control unit 260, which will later be described, and has the movement member 248 installed thereon. The movement member 248 penetrating the supporting surface of the support plate 241 protrudes from the solenoid valve 247. The movement member 248 opens or closes the conduit line of the transfer tube 5 while advancing and retreating.

That is to say, when the movement member 248 retreats, a front end of the movement member 248 extends in parallel with the supporting surface of the support plate 241 so as to sufficiently open the conduit line of the transfer tube 5, thereby enabling infusion of the Ringer's solution. When the movement member 248 advances, the transfer tube 5 is compressed by the second bending member 244 and the movement member 248 so as to close the conduit line of the transfer tube 5, thereby blocking infusion of the Ringer's solution.

In this embodiment, the movement member 248 has the solenoid valve 247 installed to be capable of advancing and retreating in a direction in which the solenoid valve 247 penetrates front and rear surfaces of the support plate 241. Alternatively, the movement member 248 may be configured to laterally pressurize the transfer tube 5. Additionally, the movement member 248 may employ a variety of types of infusion blocking units as long as they can break infusion of the Ringer's solution by closing the conduit line of the transfer tube 5.

A battery 250 for supplying power to drive the Ringer's solution infusion controlling apparatus 200 is installed in the support plate 241, and a plurality of indicator lamps 251 for indicating an amount of power of the battery 250 are provided on the front surface of the support plate 241. It is possible to determine whether the battery 250 needs to be charged or not using the number of the indicator lamps 251 turned on.

The battery 250 is preferably a rechargeable secondary battery.

In addition, a camera 252 is installed in the support plate 241 to photograph a patient's condition, and a photographing lamp 253 is formed at one side of the camera 252 to allow a patient or a caregiver for the patient to distinguish that photographing is progressed by the camera 252. A call button 254 is provided in the support plate 241 to allow the patient or the caregiver to call a nurse when necessary.

The control unit 260 is also installed in the support plate 241. The transmission signal sent from the sensor 221, the image photographed by the camera 252, and the call signal from the call button 254 are transmitted to the control unit 260. As described above, the control unit 260 transmits a driving signal for driving the infusion blocking unit 246, and includes a transmitter 261 transmits the transmitted signals to the display unit 270 to be described later.

The display unit 270 is installed at a nurse station, such as a nurse room, includes a receiver 271 receiving the signals transmitted from the control unit 260, and displays the received signals in video and/or audio signals.

That is to say, when the level of the Ringer's solution being infused reaches a preset level, a signal indicating completion of infusion of the Ringer's solution and a pertinent patient's serial number are displayed, and the image photographed by the camera 252 is displayed on an LCD window 272.

The display unit 270 receives and displays a variety of information pieces transmitted from multiple control units 260 and includes a select button 273 formed at its one side to selectively display a transmission signal from a particular one of the control units 260.

The display unit 270 may be installed in the nurse room. Alternatively, the display unit 270 may be fabricated in a portable terminal type so as to allow a nurse to carry.

The Ringer's solution infusion controlling apparatus 200 according to the present invention operates as follows.

First, the Ringer's solution storage container 1 is hung on the fixing protrusion 214, the transfer tube 5 is installed such that a predetermined region thereof is allowed to pass through the tube support unit 242, and the injection needle 30 is inserted into the patient's blood vessel to inject the Ringer's solution.

Next, the sensor 221 is set at a desired position by moving the same along the sliding hole 216. When a large amount of the Ringer's solution is to be infused, the Ringer's solution should be infused until the level of the Ringer's solution is lowered. Thus, the sensor 221 is fixed at a lower portion of the vertical panel 212. When a small amount of the Ringer's solution is to be infused, the sensor 221 is fixed at an upper portion of the vertical panel 212.

If the level of the Ringer's solution reaches where the sensor 221 is installed, a signal indicating that the level of the Ringer's solution has reached where the sensor 221 is installed, is transmitted to the control unit 260, the signal. The control unit 260 drives the infusion blocking unit 246 to make the movement member 248 advance and retreat, thereby transmitting a signal indicating of completion of infusion of the Ringer's solution by the transmitter 261 while blocking infusion of the Ringer's solution.

Then, the nurse is notified through the display unit 270 that infusion of the Ringer's solution into a pertinent patient is completed, and takes proper follow-up measures.

In addition, the nurse may monitor the patient's condition through the display unit 270 at any time. That is to say, since the camera 252 photographs the patient's surroundings and transmits image information to the display unit 270, the nurse can monitor the patient's condition at any time without checking the patient's room in person.

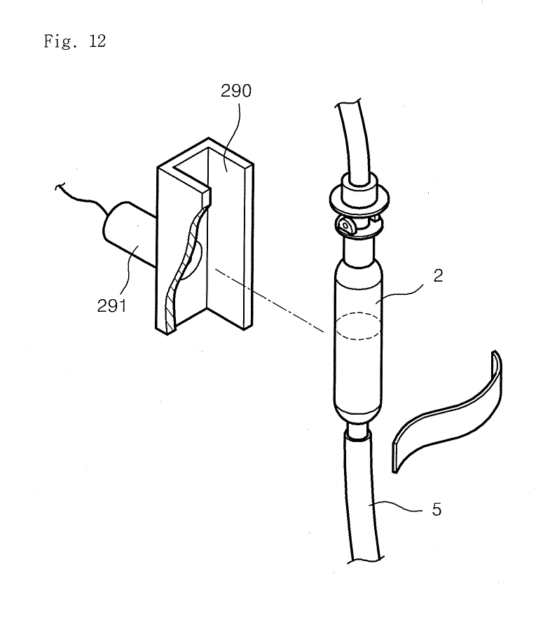

FIG. 12 illustrating another embodiment of a sensor capable of detecting that infusion of a Ringer's solution is completed.

If the Ringer's solution storage container 1 is a glass bottle, it is difficult to detect a level of the Ringer's solution using a general capacitive sensor.

In such a case that the level of the Ringer's solution stored in the Ringer's solution storage container 1 cannot be directly detected, a sensor 291 may be installed to detect the level of the Ringer's solution at the chamber 2 installed under the Ringer's solution storage container 1. That is to say, the sensor 291 is installed at one side of a chamber fixing unit 290 surrounding and fixing the chamber 2 to detect the level of the Ringer's solution remaining in the chamber 2.

When the Ringer's solution is completely infused, no Ringer's solution is induced into the chamber 2. Thus, the level of the Ringer's solution in the chamber 2 is gradually lowered. The lowering of the level of the Ringer's solution is detected by the sensor 291, suggesting that infusion of the Ringer's solution is completed.

In this case, although not illustrated, the support plate 241 may be installed on the hanger pole 3 or a stationary body around the patient's bed, the infusion blocking unit 246 opening or closing the conduit line of the transfer tube 5 is provided in the support plate 241, thereby automatically blocking infusion of the Ringer's solution.

In addition, although not illustrated, a contact sensor is attached to one side of the injection needle 30 to detect a contact state between the injection needle 30 and the patient's skin. If the injection needle 30 is extracted from the patient's body, the nurse may be notified of the extraction of the injection needle 30.

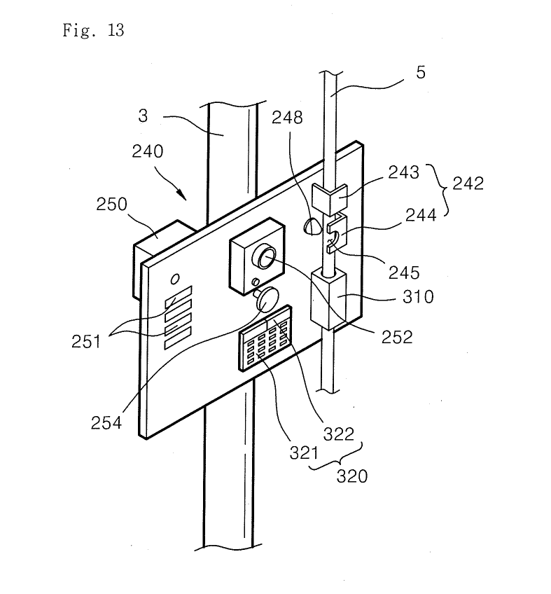

FIG. 13 illustrating another embodiment of a second unit including an infusion speed adjusting device that can automatically adjust an infusion speed of the Ringer's solution.

In a support plate 241 of the present embodiment is installed an electromechanical valve 310 as the infusion speed adjusting device that adjusts an amount of the Ringer's solution infused per unit time by dilating or shrinking a conduit line of a transfer tube 5.

The electromechanical valve 310 is formed of a solenoid valve type and is configured to dilate or shrink a conduit line of a tube. In addition, an input unit 320 is provided at one side of the support plate 241 for inputting driving information to control an infusion time or speed of the Ringer's solution. The input unit 320 consists of multiple buttons 321 to input driving information, and a display window 322 for displaying input information.

The driving information may be input in a variety of types. For example, if the amount of the Ringer's solution infused into a patient is input by a dose per time, the control unit 260 drives the electromechanical valve 310 to open the conduit line of the transfer tube 5 at a rate corresponding to the infusion speed.

If the storage capacity of the Ringer's solution storage container 1 is input and the infusion time of the Ringer's solution is then input, the control unit 260 controls the infusion speed such that the Ringer's solution in the Ringer's solution storage container 1 is completely infused for the duration of the input infusion time.

The infusion speed adjusting device that dilates or shrinks the conduit line of the transfer tube 5 to adjust the infusion speed of the Ringer's solution may take the type shown in FIG. 13, in addition to the solenoid valve.

The Ringer's solution transfer tube 5 generally includes a manual regulator that manually adjusts the infusion speed. The infusion speed adjusting device is configured to shrink the size of a conduit line by allowing a pressing roll 332 installed on a housing 331 surrounding the transfer tube 5 to pressurize the transfer tube 5 and moving the pressing roll 332.

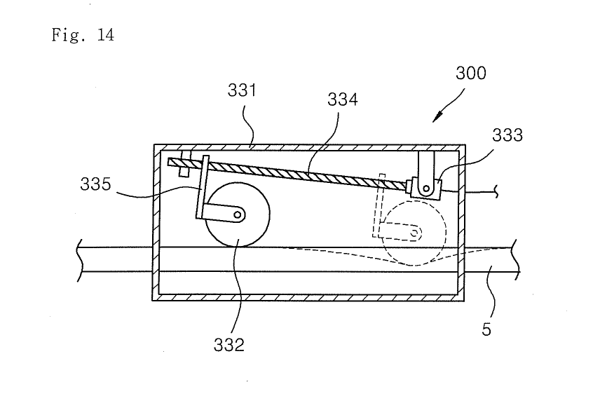

Referring to FIG. 14, an infusion speed adjusting device 300 includes a housing 331 coupled to one side of the transfer tube 5, and a pressing roll 332 coupled to the housing 331 so as to be slidable and shrinking a conduit line of the transfer tube 5 by increasingly pressurizing the transfer tube 5 from one side to the other side of the housing 331.

A driving motor 333 and a pressing roll 332 that is rotatably driven by the driving motor 333 are provided within the housing 331. The pressing roll 332 is coupled to a sliding bracket 335 threaded with a screw 334.

Therefore, if the control unit 260 drives the driving motor 333 to then rotate the screw 334, positions of the pressing roll 332 vary while the sliding bracket 335 threaded with the screw 334 moves along the screw 334.

As described above, since the Ringer's solution infusion controlling apparatuses according to the embodiments of the present invention include Ringer's solution infusion speed adjusting devices, the infusion speed, infusion amount, infusion time of the Ringer's solution can be adjusted in various manners, thereby achieving optimum infusion of the Ringer's solution so as to be best suited to the patient.

### INDUSTRIAL APPLICABILITY

As described above, the Ringer's solution injection controlling apparatus according to the present invention is very useful industrially as it is expected to be in demand from hospitals.

Although exemplary embodiments of the present invention have been described in detail hereinabove, it should be understood that many variations and modifications of the basic inventive concept herein described, which may appear to those skilled in the art, will still fall within the spirit and scope of the exemplary embodiments of the present invention as defined by the appended claims.

What is claimed is:

1. A Ringer solution infusion controlling apparatus comprising:
a body coupled to a chamber receiving a Ringer's solution from a Ringer bottle in which the Ringer's solution is stored and ejecting the Ringer's solution in a state in which the Ringer's solution is maintained at a constant level by first coupling means;
a sensing ball provided in the chamber and floating on the Ringer's solution, and elevating according to the level of the Ringer's solution in the chamber; an elevation sensor installed in the body and sensing elevation of the sensing ball;
a Ringer's solution transfer blocking unit installed in the body and having a Ringer hose passing therethrough, the Ringer hose allowing the Ringer's solution ejected from the chamber to be transferred, and blocking transfer of the Ringer's solution by compressing the Ringer hose when it is detected by the elevation sensor that the sensing ball deviates from a predetermined range; and
a wireless transmitter installed in the body and wirelessly transmitting a Ringer's solution transfer blocking signal to a nurse's room when the transfer of the Ringer's solution is blocked by the Ringer's solution transfer blocking unit.

2. The Ringer solution infusion controlling apparatus of claim 1, wherein the Ringer's solution transfer blocking unit comprises:
electromagnets positioned in the body to be opposed to each other via the body with the Ringer hose disposed therebetween, installed to be slidable toward the Ringer hose via the body, receiving external power to generate a magnetic force, sliding toward the Ringer hose by an attraction and pressing the Ringer hose;
a control unit receiving a detection signal from the elevation sensor to detect presence or absence of the sensing ball within the predetermined range and selectively supply the external power to the electromagnets; and
an elastic member installed in the body and allowing the electromagnets to be restored into their original positions by an elastic force, when supply of the external power to the electromagnets is blocked.

3. The Ringer solution infusion controlling apparatus of claim 2, further comprising:
second coupling means provided above the body and coupling and fixing the body to a supply hose supplying the chamber with the Ringer's solution from the Ringer bottle;
a speaker installed in the body, controlled by the control unit having received a detection signal from the elevation sensor, the detection signal indicating whether the sensing ball is within a predetermined range or not, and selectively generating an warning sound;
a first storage battery installed in the body and supplying power to the wireless transmitter, the electromagnets, and the speaker;
a second storage battery positioned outside the body, connected to a power supply jack installed in the body and supplying power to the wireless transmitter, the electromagnets, the speaker;
a residual power measuring unit installed in the body and measuring residual power of the first storage battery;
an indicator lamp controlled by the control unit, and when a residual power level of the first storage battery, measured by the residual power measuring unit, is a predetermined value, receiving power from the second storage battery and emitting light to indicate power consumption of the first storage battery; and
a blocking switch installed in the body and forcibly blocking power supply from the first and second storage batteries to the electromagnets,
wherein when a residual power level of the first storage battery, measured by the residual power measuring unit, is a predetermined value, the control unit receives a detection signal from the elevation sensor, the detection signal indicating whether the sensing ball is within a predetermined range or not, and controls the power from the second storage battery to be selectively supplied to the wireless transmitter, the electromagnets, the speaker.

4. A Ringer solution infusion controlling apparatus comprising:
a first unit supporting a Ringer's solution storage container and including a sensor sensing an amount of the Ringer's solution stored in the Ringer's solution storage container;
a second unit connected to the Ringer's solution storage container to support a predetermined region of a transfer tube for transferring the Ringer's solution and including an infusion blocking unit blocking movement of the Ringer's solution passing through the transfer tube;
a control unit receiving sensing information of the sensor, driving the infusion blocking unit and including a transmitter transmitting the sensing information of the sensor and a signal to indicate whether the infusion blocking unit is driven or not; and
a display unit installed in an administration room or a nurse room and including a receiver receiving the transmission signal of the transmitter and a display indicating a signal received from the receiver.

5. The Ringer solution infusion controlling apparatus of claim 4, wherein the first unit includes a hanger unit having a fixing protrusion formed at one side of an upper portion of the hanger unit to hang the Ringer's solution storage container, the sensor is installed in the hanger unit to be movable up and down to detect a level of the Ringer's solution contained in the Ringer's solution storage container hung on the hanger unit to set an infusion amount of the Ringer's solution, the second unit includes a support plate, and a tube support unit installed in the support plate and supporting a predetermined region of the transfer tube so as to extend in a linearly movable state, the infusion blocking unit is a solenoid valve blocking movement of the Ringer's solution by shutting down the conduit line of the transfer tube supported on the tube support unit.

6. The Ringer solution infusion controlling apparatus of claim 5, wherein the support plate further includes a camera that photographs a patient and patient's surroundings to monitor the patient's condition by images through the display unit, and a call button provided to allow the patient to call a nurse, and the photographed image from the camera and a call signal from the call button are transmitted to the display unit through the control unit.

7. The Ringer solution infusion controlling apparatus of claim 6, wherein the hanger unit has a sliding hole extending up and down and penetrating inner and outer surfaces thereof contacting the Ringer's solution storage container hung on the fixing protrusion; and
the sensor comprises:
an inner cylinder member including a first cylinder unit passing through the sliding hole and extending and having a hollow lengthwise and a first fixing flange provided at one end of the first cylinder unit and having an outer diameter greater than a width of the sliding hole to have a surface contacting the inner surface of the hanger unit;
a screw coupling member threaded with the hollow of the inner cylinder member;
a sensor member extending from the screw coupling member toward the inside of the hanger unit and detecting a level of the Ringer's solution stored in the Ringer's solution storage container;
an extension rod extending from the screw coupling member toward the outside of the hanger unit;
an outer cylinder member including a second cylinder unit slidably surrounding the first cylinder unit from the outside of the hanger unit, a second fixing flange provided at one end of the second cylinder unit and having a surface contacting the outer surface of the hanger unit, and a cover unit coupled to the other end of the second cylinder unit and having a passing-through hole through which the extension rod can pass;
a support flange formed at an end of the extension rod extending through the cover unit and protruding; and having an outer diameter larger than that of the extension rod; and
an elastic member having one and the other ends supported by the support flange and the cover unit, respectively and installed to surround the extension rod to be elastically biased so as to pressurize outer and inner surfaces of the hanger unit, thereby fixing the inner cylinder member and the outer cylinder member on the hanger unit hat the first and second fixing flanges.
